(19) United States
(12) Patent Application Publication
(10) Pub. No.: US 2012/0233746 A1
(43) Pub. Date: Sep. 20, 2012

(54) SNOWBOARD AND SKI HEAD PROTECTOR

(76) Inventor: Seung Jin JWA, Glendale, CA (US)

(21) Appl. No.: 13/047,423

(22) Filed: Mar. 14, 2011

(51) Int. Cl. A42B 3/12; A42C 5/04; A61F 9/02

(52) U.S. Cl. 2/414

(57) ABSTRACT

A head protector includes an inner lining member, one or more shock-absorbing pads, an outer lining member, and a band accepting groove. The one or more shock-absorbing pads are disposed on outer surface portion of the inner lining member. The one or more shock-absorbing pads are installed between the inner lining member and the outer lining member. The band accepting groove is provided on the outer surface portion of the outer lining member so as to accept and retain a band of a goggle therein. The inner surface portion of the inner lining member is contoured to fit a portion of rear head of a user so as to protect the user's head from external shock. The head protector is configured to be held against the rear head of the user wearing the goggle through the band of the goggle.

### BACKGROUND OF THE INVENTION

The present invention relates to a head protector for snowboard or ski activity.

Since the representative sports, snowboarding and skiing, are dangerous, people are recommended to wear many protecting gears.

Among those gears, a helmet is essential because the head is the most important and at the same time weakest body part.

However, conventional helmet is very bulky and hard to put on and use, and many young adults go without it under many circumstances.

Therefore, it is desirable to provide a new style of head protecting gear to the sports community, which can be effective in protecting and attractive to the young people.

Accordingly, a need for a new head protector has been present for a long time considering the expansive demands in the community. This invention is directed to solve these problems and satisfy the long-felt need.

### SUMMARY OF THE INVENTION

The present invention contrives to solve the disadvantages of the prior art. Furthermore, the invention provides a structure that has never been suggested by prior arts.

An object of the invention is to provide a head protector for snowboard and ski.

Another object of the invention is to provide a head protector, which is easily installed.

Still another object of the invention is to provide a head protector, which has simple structure.

An aspect of the invention provides a head protector comprising:

an inner lining member having an inner surface portion, an outer surface portion, and an edge portion;

one or more shock-absorbing pads disposed on the outer surface portion of the inner lining member,

an outer lining member covering the one or more shock-absorbing pads and the inner lining member and having an inner surface portion, an outer surface portion, and an edge portion, wherein the one or more shock-absorbing pads are installed between the inner lining member and the outer lining member; and

a band accepting groove provided on the outer surface portion of the outer lining member so as to accept and retain a band of a goggle therein,

wherein the inner surface portion of the inner lining member is contoured to fit a portion of rear head of a user so as to protect the user's head from external shock,

wherein the head protector is configured to be held against the rear head of the user wearing the goggle through the band of the goggle.

The inner lining member, the one or more shock-absorbing pads, may be the outer lining member are integrated into one body.

The integrated body may be flexible.

Each of the inner lining member and the outer lining member may comprise fabric.

Each of the one or more shock-absorbing pads may comprise polymer material. The polymer material may be plastic.

The shape and size of each shock-absorbing pads may be determined according to local curvature of the read head of the user and relative position in the head protector.

The head protector may further comprise:

a band cover, for covering and holding in place the goggle band, provided on the outer surface of the outer lining member and having an inner surface, an outer surface, an upper edge, a lower edge, two side edges, wherein the upper edge is fixed to the outer surface portion of the outer lining member above the band accepting groove; and

one or more first fasteners provided between the inner surface of the band cover and the outer surface portion of the outer lining member so as to hold the band cover in place disengageably.

The outer lining member and the band cover may provide a first visual pattern thereover.

Each of the one or more first fasteners may comprise a loop and hook fastener.

The head protector may further comprise:

a shell member configured to cover a portion of the outer lining member for providing more protection;

one or more second fasteners provided between an inner surface of the shell member and the outer surface portion of the outer lining member so as to hold shell member in place disengageably.

The shell member may provide a second visual pattern on an outer surface thereof.

Each of the one or more second fasteners may comprise a loop and hook fastener.

The shell member may comprise a slit for accepting and holding strands of hair of the user.

The advantages of the present invention are: (1) the head protector is easy to install; (2) the head protector is much smaller and simpler than a helmet; and (3) the head protector provides several layers of protection to the user's rear head.

Although the present invention is briefly summarized, the fuller understanding of the invention can be obtained by the following drawings, detailed description and appended claims.

### BRIEF DESCRIPTION OF THE DRAWINGS

These and other features, aspects and advantages of the present invention will become better understood with reference to the accompanying drawings, wherein:

FIG. 1 is a perspective view showing a side of a user wearing a head protector with a few shock-absorbing pads exposed and emphasized for showing purpose according to an embodiment of the invention;

FIG. 2 is a rear plan view showing the head protector in FIG. 1 with a few shock-absorbing pads exposed and emphasized for showing purpose;

FIG. 3 is a cross-sectional view showing a head protector with an inner lining member and shock-absorbing pads only for showing purpose according to an embodiment of the invention;

FIG. 4 is another cross-sectional view showing a head protector with inner and outer lining members, shock-absorbing pads, and a band cover according to an embodiment of the invention;

FIG. 5 is a rear plan view of a head protector according to an embodiment of the invention;

FIG. 6 is a front plan view showing a shell member according to an embodiment of the invention;

FIG. 7 is a rear plan view showing a shell member installed on a outer lining member according to an embodiment of the invention;

FIG. 8 is a cross-sectional view of FIG. 7;

FIG. 9 is a rear plan view showing a shell member having a slit according to an embodiment of the invention;

FIG. 10 is a perspective view showing a side of a user wearing a head protector with a partial cross-sectional view for outer lining member and shell member according to another embodiment of the invention;

FIG. 11 is a rear plan view showing the head protector in FIG. 10 with a few shock-absorbing pads exposed and emphasized for showing purpose;

FIG. 12 is another cross-sectional view showing a head protector with inner and outer lining members, shock-absorbing pads, and a band cover according to another embodiment of the invention;

FIG. 13 is a cross-sectional view of FIG. 12;

FIG. 14 is a rear plan view of a head protector according to still another embodiment of the invention; and

FIG. 15 is a rear plan view showing a shell member having a slit according to still another embodiment of the invention.

### DETAILED DESCRIPTION EMBODIMENTS OF THE INVENTION

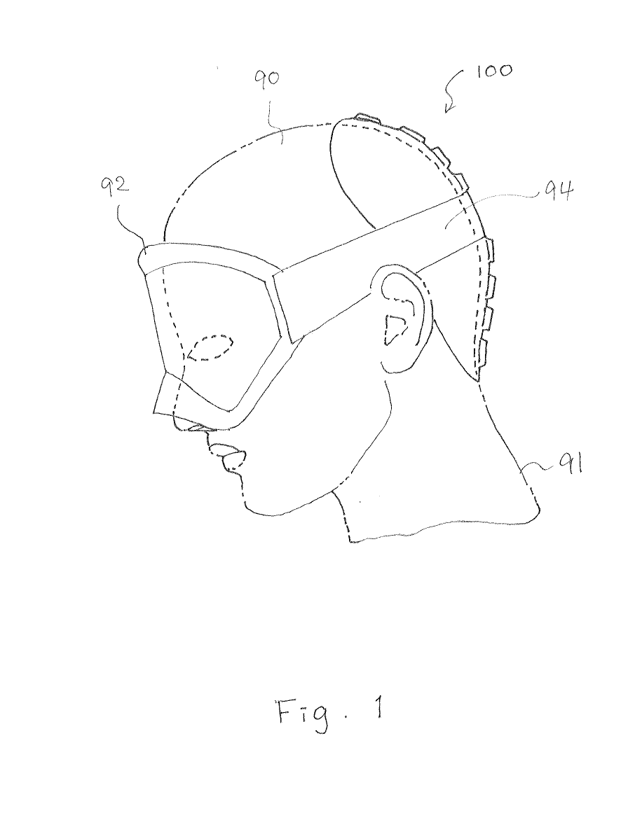

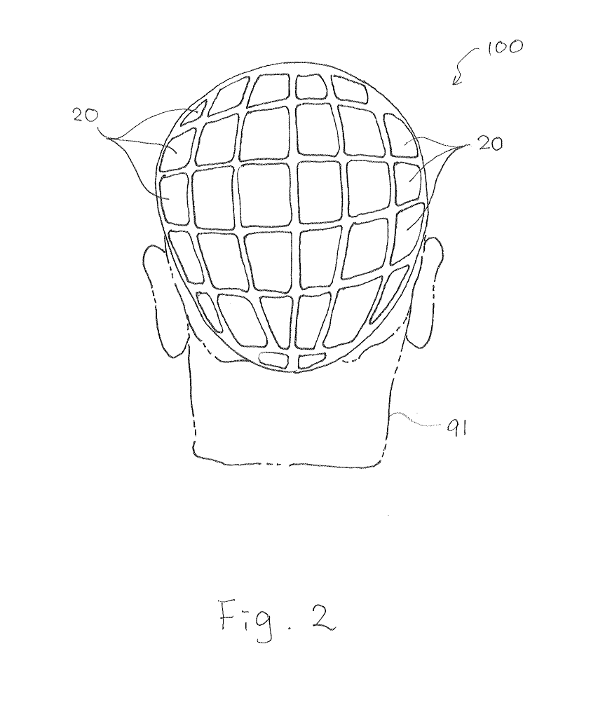

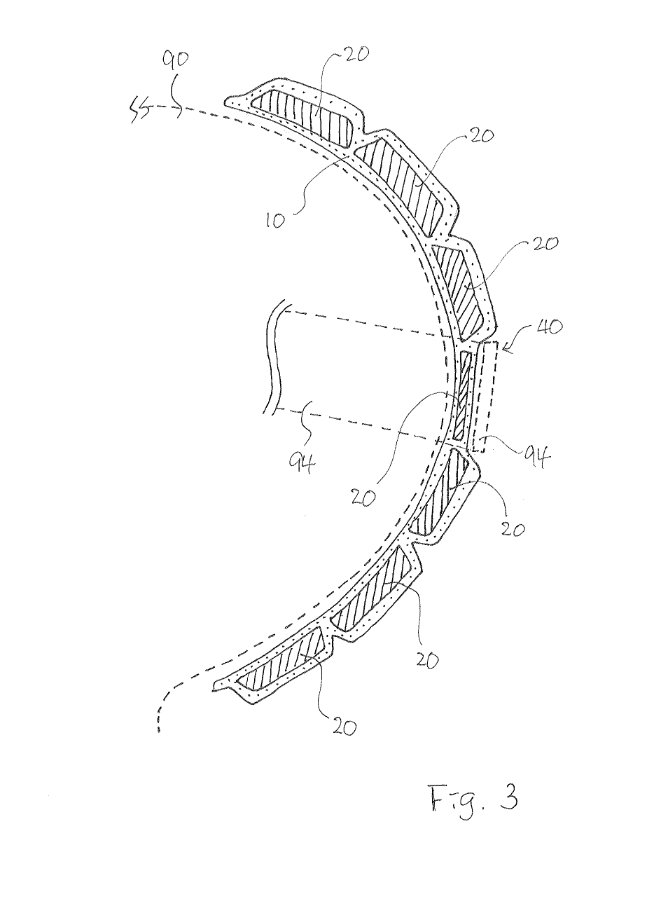

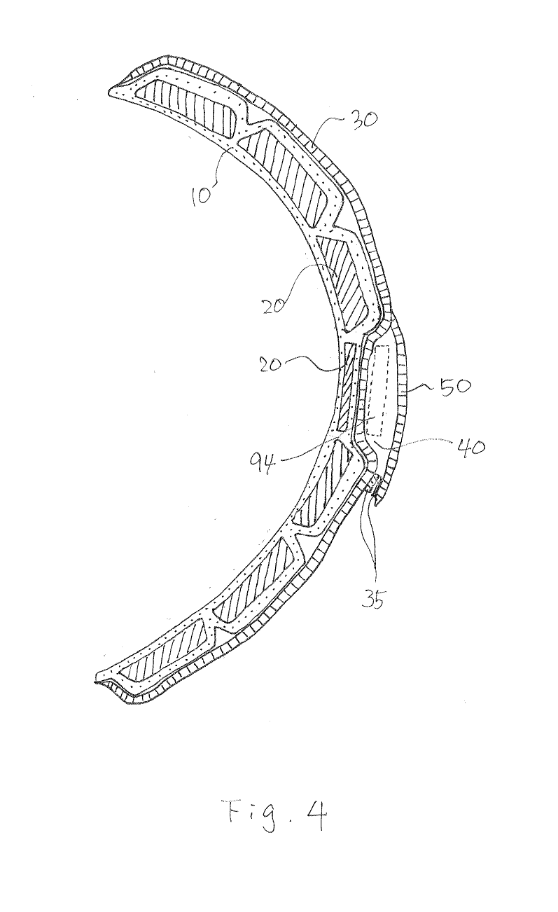

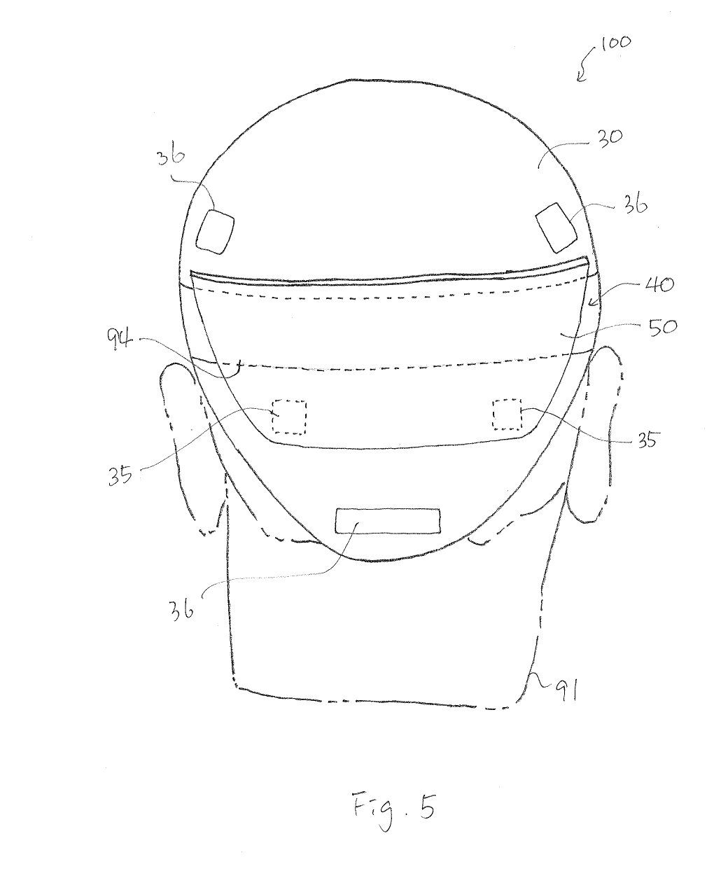

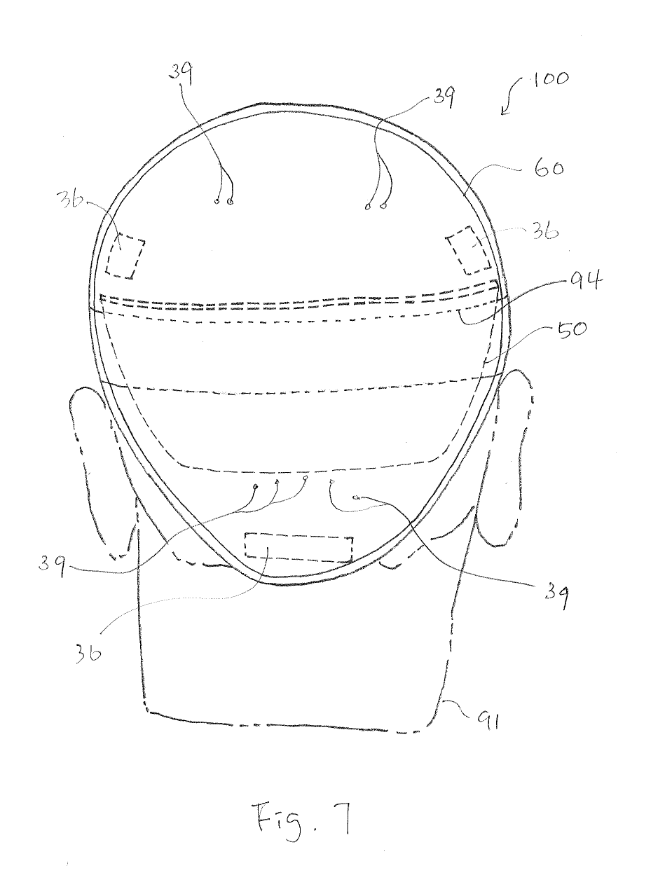

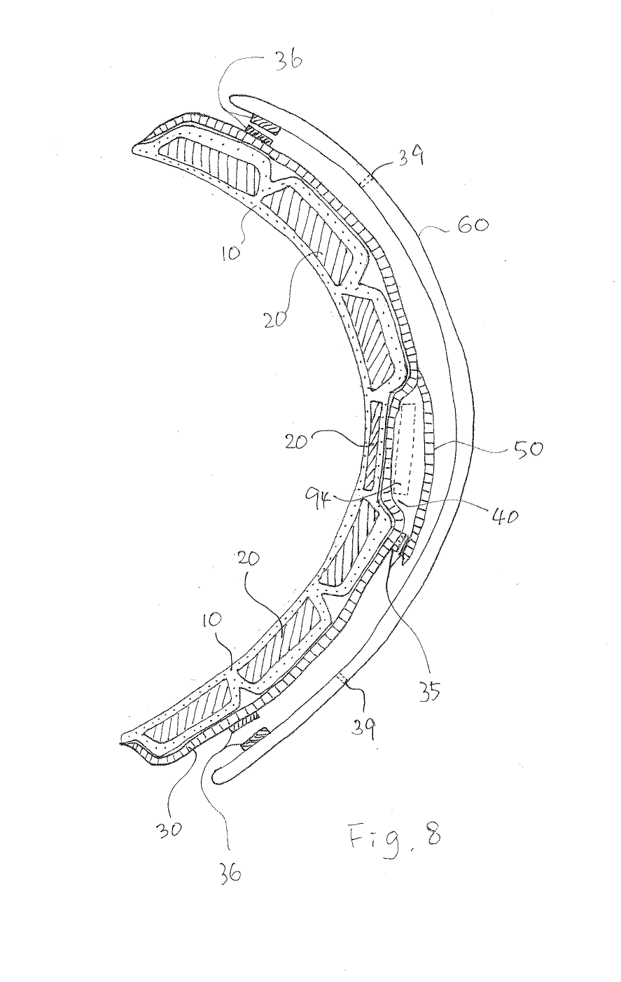

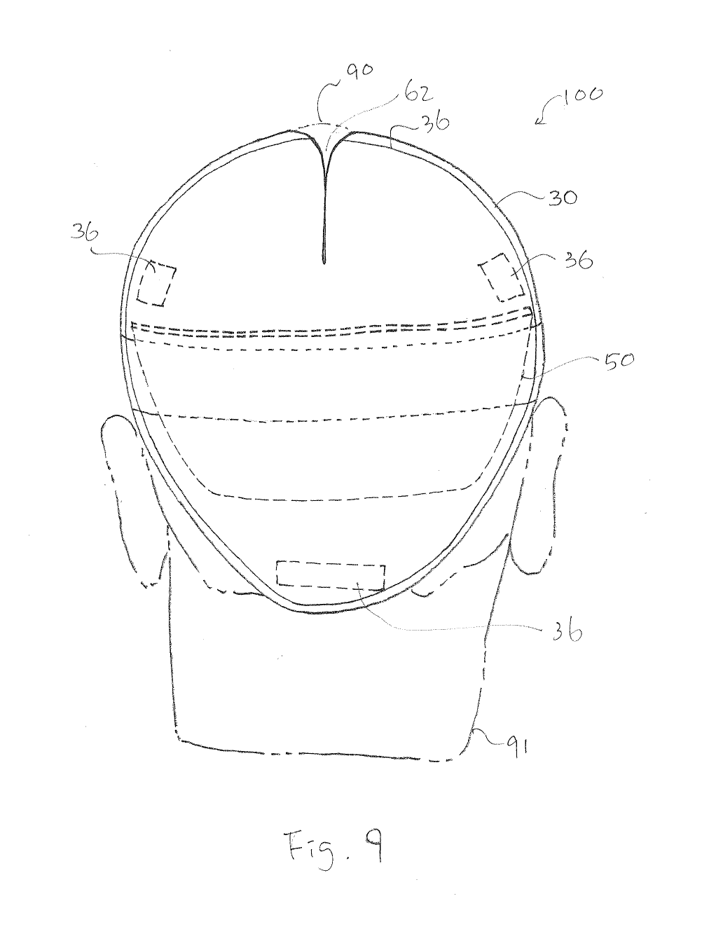

FIGS. 1-9 show a part of or complete head protectors according to embodiments of the invention.

An embodiment provides a head protector 100 comprising an inner lining member 10, one or more shock-absorbing pads 20, an outer lining member 30, and a band accepting groove 40.

The inner lining member 10 has an inner surface portion, an outer surface portion, and an edge portion.

The one or more shock-absorbing pads 20 are disposed on the outer surface portion of the inner lining member 10.

The outer lining member 30 covers the one or more shock-absorbing pads 20 and the inner lining member 10 and has an inner surface portion, an outer surface portion, and an edge portion, and the one or more shock-absorbing pads 20 are installed between the inner lining member 10 and the outer lining member 30.

The band accepting groove 40 is provided on the outer surface portion of the outer lining member 30 so as to accept and retain a band 94 of a goggle 92 therein.

The inner surface portion of the inner lining member 10 is contoured to fit a portion of rear head 90 of a user 91 so as to protect the user's head 90 from external shock.

The head protector 100 is configured to be held against the rear head 90 of the user 91 wearing the goggle 92 through the band 94 of the goggle 92.

The inner lining member 10, the one or more shock-absorbing pads 20, and the outer lining member 30 may be integrated into one body.

The integrated body may be flexible.

Each of the inner lining member 10 and the outer lining member 30 may comprise fabric.

Each of the one or more shock-absorbing pads 20 may comprise polymer material. The polymer material may be plastic.

The shape and size of each shock-absorbing pads 20 may be determined according to local curvature of the read head 90 of the user 91 and relative position in the head protector 100.

The head protector 100 may further comprise:

a band cover 50, for covering and holding in place the goggle band 94, provided on the outer surface of the outer lining member 30 and having an inner surface, an outer surface, an upper edge 52, a lower edge, two side edges, wherein the upper edge 52 is fixed to the outer surface portion of the outer lining member 30 above the band accepting groove 40; and

one or more first fasteners 35 provided between the inner surface of the band cover 50 and the outer surface portion of the outer lining member 30 so as to hold the band cover 50 in place disengageably.

The outer lining member 30 and the band cover 50 may provide a first visual pattern (not shown) thereover.

Each of the one or more first fasteners 35 may comprise a loop and hook fastener such as Velcro®.

The head protector 100 may further comprise:

a shell member 60 configured to cover a portion of the outer lining member 30 for providing more protection;

one or more second fasteners 36 provided between an inner surface of the shell member 60 and the outer surface portion of the outer lining member 30 so as to hold shell member 60 in place disengageably.

The shell member 60 may provide a second visual pattern (not shown) on an outer surface thereof.

Each of the one or more second fasteners 36 may comprise a loop and hook fastener including Velcro®.

The shell member 60 may further comprise a slit 62 for accepting and holding strands of hair of the user. The slit 62 may be provided at an upper portion of the shell member 60. The material around the slit 62 may be elastic, such that the hair strand may be inserted easily.

In certain embodiment of the invention, the location and size of the first or second fasteners 35, 36 may be determined to make the fastening secure.

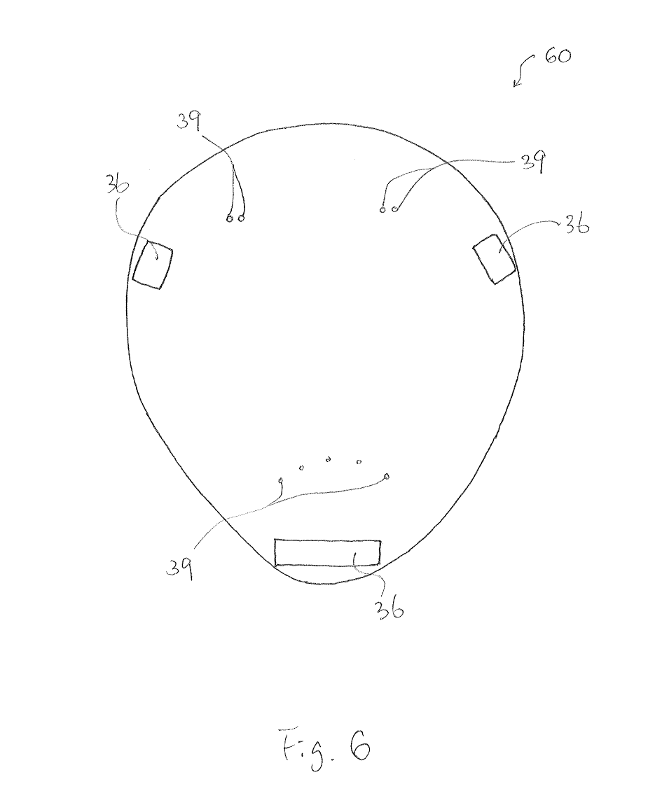

The shell member 60 may further comprise a plurality of ventilation holes 39 penetrated therethrough as shown in FIGS. 6-8. The ventilation holes 39 help air exchange between both sides of the shell member 60 for cooling down inside.

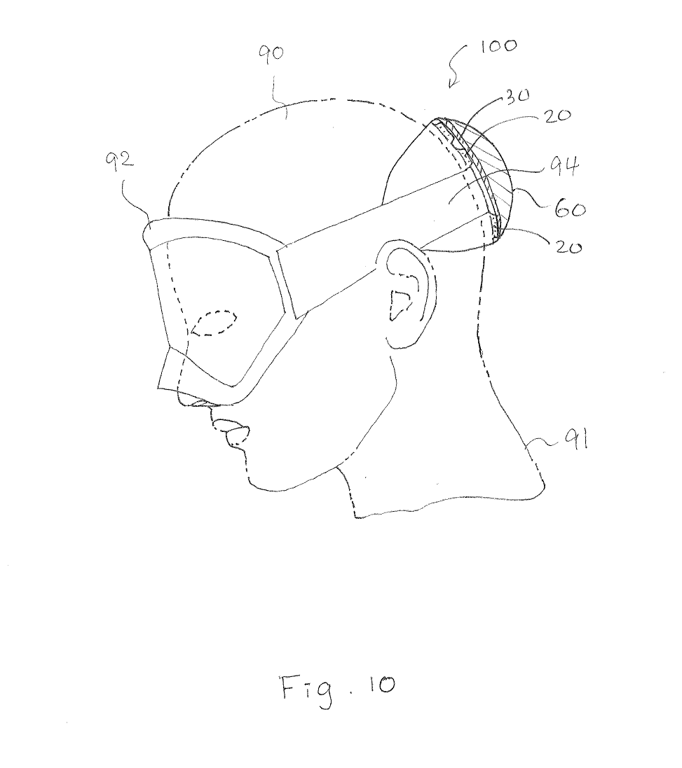

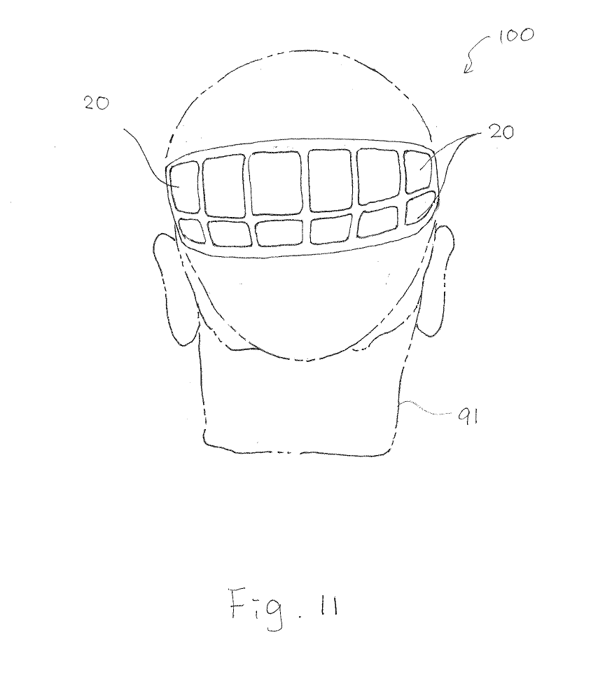

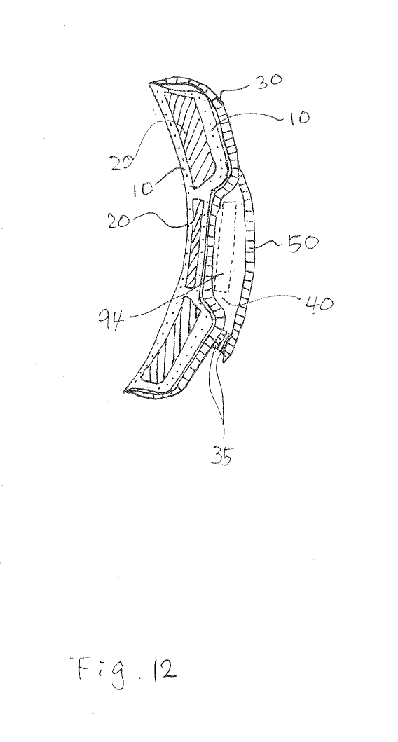

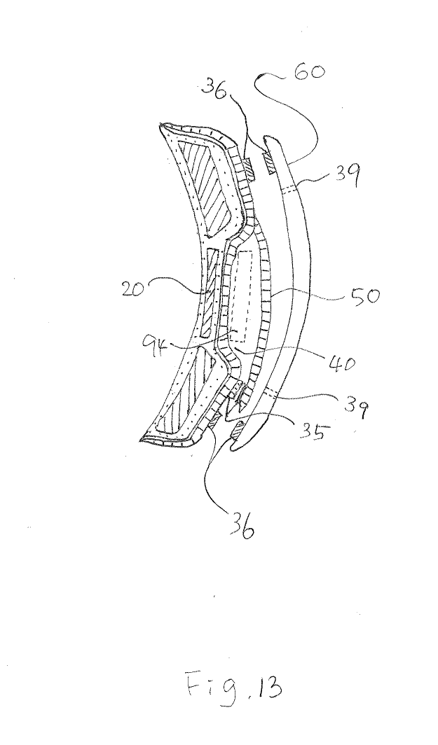

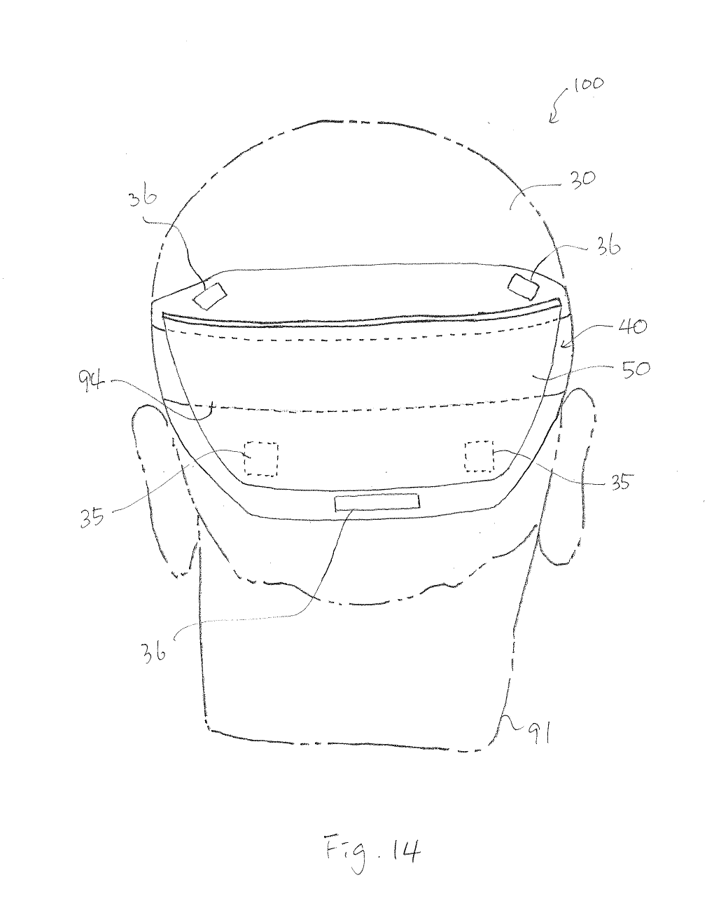

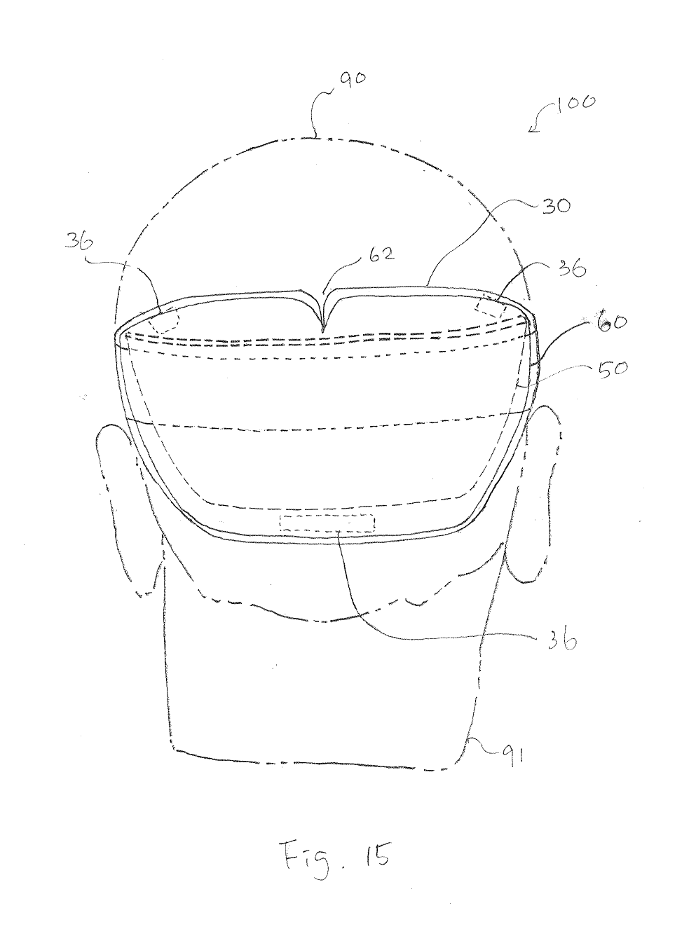

FIGS. 10-15 show another head protector according to another embodiment of the invention.

The size of the head protector 100 is reduced vertically in order to obtain a different or hopefully improved style. Since it is smaller than that in FIGS. 1-9, the head protector 100 may be easier to wear and maintain.

Especially, the shell member 60 has a little more bulged shape to increase shock-absorbing capability or style. As shown in this embodiment, the exact size of shape may of the parts may be modified without leaving the spirit of the invention.

The material and function of the parts in FIGS. 10-15 may be similar to those of the corresponding parts in FIGS. 1-9.

While the invention has been shown and described with reference to different embodiments thereof, it will be appreciated by those skilled in the art that variations in form, detail, compositions and operation may be made without departing from the spirit and scope of the invention as defined by the accompanying claims.

What is claimed is:

1. A head protector comprising:
an inner lining member having an inner surface portion, an outer surface portion, and an edge portion;
one or more shock-absorbing pads disposed on the outer surface portion of the inner lining member,
an outer lining member covering the one or more shock-absorbing pads and the inner lining member and having an inner surface portion, an outer surface portion, and an edge portion, wherein the one or more shock-absorbing pads are installed between the inner lining member and the outer lining member; and
a band accepting groove provided on the outer surface portion of the outer lining member so as to accept and retain a band of a goggle therein,
wherein the inner surface portion of the inner lining member is contoured to fit a portion of rear head of a user so as to protect the user's head from external shock,
wherein the head protector is configured to be held against the rear head of the user wearing the goggle through the band of the goggle.

2. The head protector of claim 1, wherein the inner lining member, the one or more shock-absorbing pads, are the outer lining member are integrated into one body.

3. The head protector of claim 2, wherein the integrated body is flexible.

4. The head protector of claim 1, wherein each of the inner lining member and the outer lining member comprises fabric.

5. The head protector of claim 1, wherein each of the one or more shock-absorbing pads comprises polymer material.

6. The head protector of claim 5, wherein the polymer material is plastic.

7. The head protector of claim 6, wherein the shape and size of each shock-absorbing pads is determined according to local curvature of the read head of the user and relative position in the head protector.

8. The head protector of claim 1, further comprising:
a band cover, for covering and holding in place the goggle band, provided on the outer surface of the outer lining member and having an inner surface, an outer surface, an upper edge, a lower edge, two side edges, wherein the upper edge is fixed to the outer surface portion of the outer lining member above the band accepting groove; and
one or more first fasteners provided between the inner surface of the band cover and the outer surface portion of the outer lining member so as to hold the band cover in place disengageably.

9. The head protector of claim 8, wherein the outer lining member and the band cover provide a first visual pattern thereover.

10. The head protector of claim 8, wherein each of the one or more first fasteners comprises a loop and hook fastener.

11. The head protector of claim 1, further comprising:
a shell member configured to cover a portion of the outer lining member for providing more protection;
one or more second fasteners provided between an inner surface of the shell member and the outer surface portion of the outer lining member so as to hold shell member in place disengageably.

12. The head protector of claim 11, wherein the shell member provides a second visual pattern on an outer surface thereof.

13. The head protector of claim 12, wherein each of the one or more second fasteners comprises a loop and hook fastener.

14. The head protector of claim 11, wherein the shell member comprises a slit for accepting and holding strands of hair of the user.

15. The head protector of claim 11, wherein the shell member comprises a plurality of ventilation holes penetrated therethrough.
